(19) United States
(12) Patent Application Publication
(10) Pub. No.: US 2025/0170415 A1
(43) Pub. Date: May 29, 2025

(54) IMPLANTABLE OPTICAL STIMULATION LEAD WITH ALUMINUM NITRIDE FOR HEAT DISSIPATION

(71) Applicant: Boston Scientific Neuromodulation Corporation, Valencia, CA (US)

(72) Inventors: Joshua Dale Howard, Sacramento, CA (US); Steven Yihlih Peng, Fremont, CA (US); Gregory Bates, Bend, OR (US)

(21) Appl. No.: 18/959,026

(22) Filed: Nov. 25, 2024

Related U.S. Application Data
(60) Provisional application No. 63/603,393, filed on Nov. 28, 2023.

(51) Int. Cl. A61N 5/06; A61N 5/067

(52) U.S. Cl. CPC A61N 5/0601; A61N 5/067; A61N 2005/0653; A61N 2005/0662

(57) ABSTRACT

An optical stimulation lead includes a lead body including a distal end, a distal portion, and a proximal portion; and an optical assembly attached to the distal end of the lead body. The optical assembly includes a metal enclosure; a light emitter disposed within the metal enclosure; an electrical feedthrough pin electrically coupled to the light emitter; a first substrate disposed within the metal enclosure; and an emitter cover disposed opposite the lead body, configured to transmit light from the light emitter, and coupled to the metal enclosure. The first substrate includes aluminum nitride and the first substrate is arranged within the metal enclosure with the aluminum nitride in a thermal conductive path that conducts heat from the light emitter to the metal enclosure.

### CROSS-REFERENCE TO RELATED APPLICATIONS

This application claims the benefit under 35 U.S.C. § 119 (e) of U.S. Provisional Patent Application Ser. No. 63/603,393, filed Nov. 28, 2023, which is incorporated herein by reference.

### FIELD

The present invention is directed to the area of implantable optical stimulation systems and methods of making and using the systems. The present invention is also directed to implantable optical stimulation leads having a light emitter and having a substrate composed of aluminum nitride that provides heat dissipation for the light emitter, as well as methods of making and using the leads and optical stimulation systems.

### BACKGROUND

Implantable optical stimulation systems can provide therapeutic benefits in a variety of diseases and disorders. For example, optical stimulation can be applied to the brain either externally or using an implanted stimulation lead to provide, for example, deep brain stimulation, to treat a variety of diseases or disorders. Optical stimulation may also be combined with electrical stimulation.

Stimulators have been developed to provide therapy for a variety of treatments. A stimulator can include a control module (for generating light or electrical signals sent to light sources in a lead), one or more leads, and one or more light sources coupled to, or disposed within, each lead. The lead is positioned near the nerves, muscles, or other tissue to be stimulated.

### BRIEF SUMMARY

One aspect is an optical stimulation lead including a lead body including a distal end, a distal portion, and a proximal portion; and an optical assembly attached to the distal end of the lead body. In at least some aspects, the optical assembly includes a metal enclosure; a light emitter disposed within the metal enclosure; an electrical feedthrough pin electrically coupled to the light emitter; a first substrate disposed within the metal enclosure; and an emitter cover disposed opposite the lead body, configured to transmit light from the light emitter, and coupled to the metal enclosure. The first substrate includes aluminum nitride and the first substrate is arranged within the metal enclosure with the aluminum nitride in a thermal conductive path that conducts heat from the light emitter to the metal enclosure.

In at least some aspects, the metal enclosure includes titanium. In at least some aspects, the light emitter includes a laser diode. In at least some aspects, the optical assembly forms a hermetically sealed housing for the light emitter. In at least some aspects, the optical assembly further includes a plurality of terminals disposed along the proximal portion of the lead body and a plurality of conductors extending along the lead body. In at least some aspects, at least one of the conductors of the plurality of conductors electrically couples the electrical feedthrough pin to at least one of the terminals of the plurality of terminals of the lead.

In at least some aspects, the emitter cover includes at least one of an optical diffuser or a lens. In at least some aspects, the optical assembly further includes a non-permeable gel disposed within the metal enclosure. In at least some aspects, the optical assembly further includes a second substrate different from the first substrate, wherein the light emitter is attached to the second substrate and the second substrate is disposed between the first substrate and the light emitter.

In at least some aspects, the optical assembly further includes a getter disposed within the metal enclosure. In at least some aspects, the getter includes a desiccant. In at least some aspects, the first substrate is disposed between the light emitter and the getter. In at least some aspects, the getter is disposed on the metal enclosure around the light emitter.

In at least some aspects, the optical assembly further includes: a non-conductive block, and a metal base disposed around the non-conductive block. In at least some aspects, the electrical feedthrough pin extends through the non-conductive block. In at least some aspects, the metal enclosure includes at least a portion of the metal base. In at least some aspects, the first substrate is in contact with the metal base.

In at least some aspects, the optical assembly further includes an adhesive, the first substrate is disposed between the light emitter and the adhesive, and the adhesive includes boron nitride. In at least some aspects, the optical assembly further includes a getter disposed within the metal enclosure, the first substrate is disposed between the light emitter and the getter, the getter includes a desiccant, and the getter at least partially surrounds the adhesive.

Another aspect is an optical stimulation system that includes any of the optical stimulation lead described above and a control module coupleable to the optical stimulation lead. In at least some aspects, the control module includes a housing and an electronic subassembly disposed in the housing and configured and arranged to provide electrical signals to the light emitter of the optical stimulation lead. In at least some aspects, the connector is part of the control module. In at least some aspects, the electrical stimulation system further includes a lead extension coupleable to both the electrical stimulation lead and the control module. In at least some aspects, the connector is part of the lead extension.

Yet another aspect is a method of stimulating patient tissue that includes implanting any of the optical stimulation lead described above into the patient tissue; and emitting light from the light emitter of the optical stimulation lead to stimulate the patient tissue.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of an optical stimulation system that includes a lead electrically coupled to a control module;

FIG. 2A is a schematic side view of one embodiment of the control module of FIG. 1 configured and arranged to couple to an elongated device;

FIG. 2B is a schematic side view of one embodiment of a lead extension configured and arranged to couple the elongated device of FIG. 2A to the control module of FIG. 1;

FIG. 3 is a schematic perspective view of one embodiment of a lead body that may be employed at one embodiment of the lead of FIG. 1;

FIG. 4 is a schematic side view of one embodiment of an optical assembly for the lead of FIG. 1 in which the optical assembly includes an aluminum nitride substrate;

FIG. 5 is a schematic side view of an embodiment of the optical assembly of FIG. 4 in which the aluminum nitride substrate directly touches a base of the optical assembly;

FIG. 6 is a schematic side view of an embodiment of the optical assembly of FIG. 4 in which a getter is arranged next to the aluminum nitride substrate;

FIG. 7A is a schematic side view of an embodiment of the optical assembly of FIG. 4 in which the aluminum nitride substrate touches the metal enclosure;

FIG. 7B is a schematic side view of an embodiment of the optical assembly of FIG. 7A in which the optical assembly further includes a getter;

FIG. 8 is a schematic side view of an embodiment of the optical assembly of FIG. 4 in which the aluminum nitride substrate is disposed next to another substrate;

FIG. 9 is a schematic side view of an embodiment of the optical assembly of FIG. 4 in which an adhesive is disposed next to the aluminum nitride substrate;

FIG. 10 is a schematic side view of an embodiment of the optical assembly of FIG. 4 in which in which an adhesive is disposed next to the aluminum nitride substrate and the adhesive circumferentially surrounded by a getter; and

FIG. 11 is a schematic overview of one embodiment of components of an optical stimulation system, including an electronic subassembly disposed within a control module.

### DETAILED DESCRIPTION

The present invention is directed to the area of implantable optical stimulation systems and methods of making and using the systems. The present invention is also directed to implantable optical stimulation leads having a light emitter and having a substrate composed of aluminum nitride that provides heat dissipation for the light emitter, as well as methods of making and using the leads and optical stimulation systems.

In some embodiments, the implantable optical stimulation system only provides optical stimulation. In other embodiments, the stimulation system can include both optical and electrical stimulation. In at least some of these embodiments, the optical stimulation system can be a modification of an electrical stimulation system to also provide optical stimulation. Suitable implantable electrical stimulation systems that can be modified to also provide optical stimulation include, but are not limited to, at least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 6,175,710; 6,224,450; 6,271,094; 6,295,944; 6,364,278; 6,391,985; 10,335,607; and 11,511,127; U.S. Patent Applications Publication Nos. 2007/0150036; 2009/0187222; 2009/0276021; 2010/0076435; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; and 2013/0105071; and U.S. patent application Ser. Nos. 12/177,823 and 13/750,725, all of which are incorporated by reference in their entireties.

Optical stimulation can include photostimulation or photobiomodulation (PBM) or both. Photostimulation can include generation of a neural signal (i.e., an electrical signal) or an action potential by the activation of neural tissue (e.g., neurons or other neural cells) in response to optical irradiation. PBM may include, but is not necessarily limited to, optical modulation, other modulation, or other effects resulting from biological response to particular wavelengths or wavelength ranges of light or from thermal effects generated using light or from any combination thereof. Unless otherwise indicated, PBM does not include photostimulation. PBM can include a biological or cellular response to optical irradiation excluding photostimulation. In at least some embodiments, the biological or cellular response includes modulation (for example, up-regulation or down-regulation) of one or more chemical or biological entities.

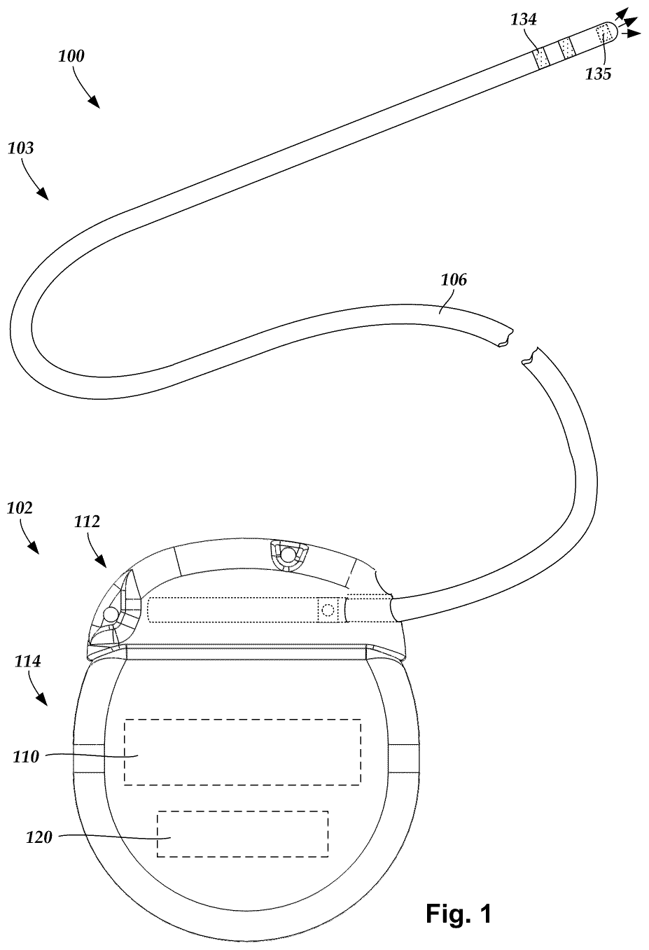

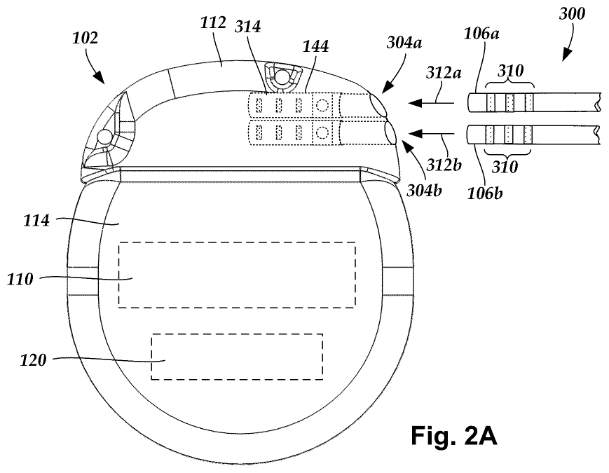

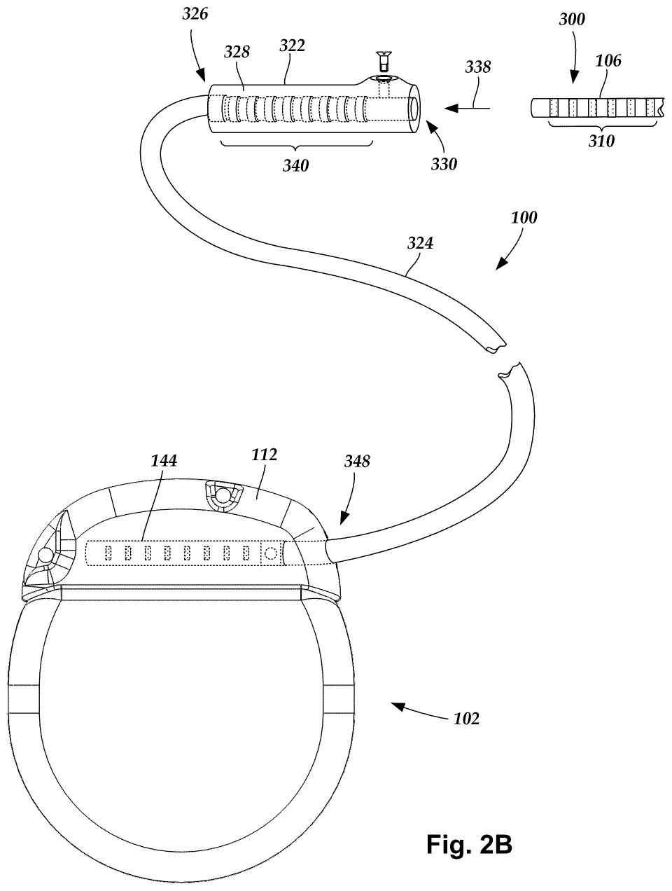

FIG. 1 illustrates schematically one embodiment of an optical stimulation system 100. The optical stimulation system includes a control module (e.g., a stimulator) 102 and a lead 103 coupleable to the control module 102. The lead 103 includes one or more lead bodies 106. In FIG. 1, the lead 103 is shown having a single lead body 106. In FIG. 2B, the lead 103 includes two lead bodies. It will be understood that the lead 103 can include any suitable number of lead bodies including, for example, one, two, three, four, five, six, seven, eight or more lead bodies 106.

At least one light emitter 135 is provided at a distal end of the lead 103. The light emitter 135 can be a light source, such as a light emitting diode (LED), laser diode, organic light emitting diode (OLED), or the like. Optionally, the lead can also include one or more electrodes 134 disposed along the lead body 106, and one or more terminals (e.g., 310 in FIG. 2A-2B) disposed along each of the one or more lead bodies 106 and coupled to the optional electrodes 134 by conductors (not shown). In at least some embodiments, one or more terminals (e.g., 310 in FIG. 2A-2B) may also be used to convey electrical signals to a light source that acts as the light emitter 135 by conductors (not shown) extending along the lead.

The lead 103 can be coupled to the control module 102 in any suitable manner. In some embodiments, the lead is permanently attached to the control module 102. In other embodiments, the lead can be coupled to the control module 102 by a connector (e.g., connector 144 of FIG. 2A). In FIG. 2A, the lead 103 is shown coupling directly to the control module 102 through the connector 144. In at least some other embodiments, the lead 103 couples to the control module 102 via one or more intermediate devices, as illustrated in FIG. 2B. For example, in at least some embodiments one or more lead extensions 324 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the stimulation system 100 includes multiple elongated devices disposed between the lead 103 and the control module 102, the intermediate devices may be configured into any suitable arrangement.

The control module 102 can include, for example, a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The stimulation system or components of the stimulation system, including the lead 103 and the control module 102, are typically implanted into the body of a patient. The stimulation system can be used for a variety of applications including, but not limited to brain stimulation, deep brain stimulation, neural stimulation, spinal cord stimulation, muscle stimulation, and the like. Optical stimulation performed by the stimulation system may include photobiomodulation, photostimulation, and other suitable forms of optical stimulation.

If the lead includes the optional electrodes 134, the electrodes can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. Any suitable number of electrodes 134 can be disposed on the lead including, for example, one, two, three, four, five, six, seven, eight, nine, ten, eleven, twelve, fourteen, sixteen, twenty-four, thirty-two, or more electrodes 134.

The one or more lead bodies 106 are made of a non-conductive, biocompatible material such as, for example, silicone, polyurethane, polyetheretherketone (“PEEK”), epoxy, and the like or combinations thereof. The one or more lead bodies 106 may be formed in the desired shape by any process including, for example, molding (including injection molding), casting, and the like.

One or more terminals (e.g., 310 in FIGS. 2A-2B) are typically disposed along the proximal end of the one or more lead bodies 106 of the stimulation system 100 (as well as any splitters, lead extensions, adaptors, or the like) for electrical connection to corresponding connector contacts (e.g., 314 in FIGS. 2A-2B). The connector contacts are disposed in connectors (e.g., 144 in FIGS. 1-2B; and 322 FIG. 2B) which, in turn, are disposed on, for example, the control module 102 (or a lead extension, a splitter, an adaptor, or the like). Electrically conductive wires, cables, or the like (not shown) extend from the terminals to the light emitter 135 or optional one or more electrodes 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 2A is a schematic side view of one embodiment of a proximal end of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 2B, an adaptor, or the like or combinations thereof), or a combination thereof.

The control module connector 144 defines at least one port into which a proximal end of the elongated device 300 can be inserted, as shown by directional arrows 312a and 312b. In FIG. 2A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 300 is inserted into the ports 304a and 304b, the connector contacts 314 can be aligned with a plurality of terminals 310 disposed along the proximal end(s) of the elongated device(s) 300 to electrically couple the control module 102 to the electrodes (134 of FIG. 1) disposed on the paddle body 104 of the lead 103. Each of the terminals 310 can couple to the light emitter 135 or one or more of the optional electrodes 134. Examples of connectors in control modules are found in, for example, U.S. Pat. Nos. 7,244,150 and 8,224,450, which are incorporated by reference.

FIG. 2B is a schematic side view of another embodiment of the stimulation system 100. The stimulation system 100 includes a lead extension 324 that is configured and arranged to couple one or more elongated devices 300 (e.g., one of the lead bodies 106 of FIG. 1, a splitter, an adaptor, another lead extension, or the like or combinations thereof) to the control module 102. In FIG. 2B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144 (e.g., the ports 304a and 304b of FIG. 1), or to receive multiple elongated devices 300 (e.g., both of the lead bodies 106 of FIG. 1), or both.

A lead extension connector 322 is disposed on the lead extension 324. In FIG. 2B, the lead extension connector 322 is shown disposed at a distal end 326 of the lead extension 324. The lead extension connector 322 includes a connector housing 328. The connector housing 328 defines at least one port 330 into which terminals 310 of the elongated device 300 can be inserted, as shown by directional arrow 338. Each of the terminals 310 can couple to the light emitter 135 or one or more of the optional electrodes 134. The connector housing 328 also includes a plurality of connector contacts, such as connector contact 340. When the elongated device 300 is inserted into the port 330, the connector contacts 340 disposed in the connector housing 328 can be aligned with the terminals 310 of the elongated device 300 to electrically couple the lead extension 324 to the electrodes (134 of FIG. 1) disposed along the lead (103 in FIG. 1).

In at least some embodiments, the proximal end of the lead extension 324 is similarly configured and arranged as a proximal end of the lead 103 (or other elongated device 300). The lead extension 324 may include a plurality of electrically conductive wires (not shown) that electrically couple the connector contacts 340 to a proximal end 348 of the lead extension 324 that is opposite to the distal end 326. In at least some embodiments, the conductive wires disposed in the lead extension 324 can be electrically coupled to a plurality of terminals (not shown) disposed along the proximal end 348 of the lead extension 324. In at least some embodiments, the proximal end 348 of the lead extension 324 is configured and arranged for insertion into a connector disposed in another lead extension (or another intermediate device). In other embodiments (and as shown in FIG. 2B), the proximal end 348 of the lead extension 324 is configured and arranged for insertion into the control module connector 144.

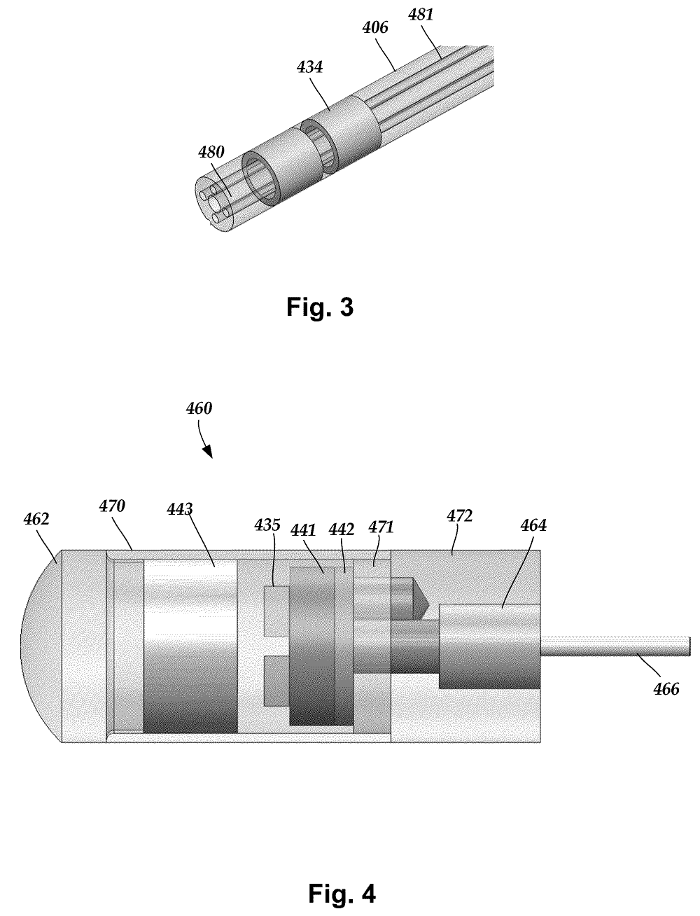

FIG. 3 illustrates a distal end of one embodiment of a lead (e.g., lead 103 of FIG. 1) that includes a lead body 406 and two optional electrodes 434 disposed along the lead body. The lead body 406 of the illustrated embodiment includes a central lumen 480 and one or more peripheral lumens 481. Other arrangements of a lead can be used including leads with more or fewer lumens.

In at least some embodiments, one or more of the peripheral lumens 481 can carry conductors (not shown), such as conductive wires or cables. In at least some embodiments, one or more electrical feedthrough pins of the optical assembly (e.g., optical assembly 460 of FIG. 4) can be inserted into one or more of the peripheral lumens 481 and can be coupled to the conductors carried in those lumens in order to transmit electrical signals from the control module through the conductors to the optical assembly. In at least some embodiments, conductors in one or more of the peripheral lumens 481 can also be coupled to the optional electrodes 434, as described above and in the references cited above.

An optical assembly can be attached to the distal end of the lead. FIG. 4 illustrates one embodiment of an optical assembly 460 for a lead 103 (FIG. 1). In FIG. 4, some elements are shown as partially transparent for illustrative purposes. In at least some embodiments, the optical assembly 460 includes at least one light emitter 435, an emitter cover 462, a substrate 441, an optional getter 442, an optional seal 443, a non-conductive block 464, at least one electrical feedthrough pin 466, a metal enclosure 470, a metal washer 471, and a metal endcap 472. Although FIG. 4 shows the getter 442 in one particular position, in various embodiments, the getter 442 may be excluded or arranged in a different position in the optical assembly 460, examples of which are discussed below in other embodiments. Any suitable number of light emitters 435 can be used, including, but not limited to, 1, 2, 3, 4, 6, 8 or more light emitters. Any suitable number of electrical feedthrough pins 466 can be used.

Other embodiments of an optical assembly 460 can include more or fewer components. For instance, in some embodiments, the optical assembly 460 does not include the seal 443 (e.g., the emitter cover 462 may act as a seal). As another example, in some embodiments, the light emitter(s) 435 are coupled with elements external to the optical assembly 460 in a suitable way other than the non-conductive block 464 and the electrical feedthrough pin(s) 466.

Each of the light emitter(s) 435 may be any suitable light emitter, including, but not limited to, a light emitting diode (LED), laser diode, organic light emitting diode (OLED), or the like. Each of the light emitter(s) 435 can emit one or more wavelengths of light or can emit light in one or more wavelength bands. The wavelength or wavelength band can be visible, infrared, or ultraviolet light. In at least some embodiments, the light emitter(s) 435 emit visible light. In at least some embodiments, the light emitter(s) 435 emit light at one or more wavelengths in a range of 500 to 800 nm or in a range of 600 to 700 nm. The light emitter(s) 435 may emit light at a same wavelength or wavelength band or some, or all, of the light emitter(s) 435 may emit light at different wavelengths or wavelength bands. In at least some embodiments, each of the light emitter(s) 435 includes an emitter element, a housing, and at least two contacts.

In at least some embodiments, an emitter cover 462 is disposed over the light emitter 435 opposite the distal end of the lead. In at least some embodiments, the emitter cover 462 is an optical component such as an optical diffuser or lens. For example, a sapphire optical diffuser dome can be positioned over the light emitter. In other embodiments, the emitter cover 462 is an optically transparent or translucent material to seal the light emitter 435 within the optical assembly 460. In some embodiments, the emitter cover 462 is part of a package that forms the light emitter(s) 435. In at least some embodiments, the seal 443 hermetically seals the emitter cover 462 to the metal enclosure 470. In at least some embodiments, the seal 443 is composed of ceramic (e.g., polycrystalline ceramic) or any other suitable material.

The electrical feedthrough pin(s) 466 are electrically coupled to the light emitter 435. Any suitable arrangement for electrical coupling the electrical feedthrough pin(s) 466 to the light emitter 435 can be used. For instance, in some embodiments, one or more electrical feedthrough pins 466 is connected to light emitter 435 through one or more holes in the substrate 441. In some embodiments, one or more electrical feedthrough pins 466 are electrically connected to light emitter 435 through one or more contacts on substrate 441, through one or more conductive vias through substrate 441, or any combination thereof or in any other suitable arrangement.

The electrical feedthrough pin(s) 466 are made of metal or any other suitable conductive material. For example, the electrical feedthrough pin(s) 466 can be made of platinum, iridium, platinum/iridium alloy, or any other suitable material. In at least some embodiments, each of the electrical feedthrough pin(s) 466 is coupled to the light emitter(s) 435 via the substrate 441, passes through one or more elements in the optical assembly 460 such as the getter 442 or the non-conductive block 464, extends outside of the optical assembly 460, and connects to a corresponding one of the peripheral lumen(s) 481 (FIG. 3). In at least some embodiments, the non-conductive block 464 includes one or more ceramic blocks through which at least one electrical feedthrough pin 466 passes. In at least some embodiments, at least a portion of the non-conductive block 464 is disposed within the metal enclosure 470. In at least some embodiments, the non-conductive block 464 is composed of ceramic (e.g., polycrystalline ceramic) or any other suitable non-conductive material.

The metal enclosure 470 is disposed around the light emitter 435. In at least some embodiments, the metal enclosure 470 is composed of titanium. In at least some embodiments, the optical assembly 460 provides a hermetically sealed housing for the light emitter 435 which prevents or resists the flow of fluids into the optical assembly 460. For example, the use of welding and brazing to couple the components of the optical assembly 460 may facilitate or provide the hermetical sealing of the optical assembly 460.

In at least some embodiments, the optical assembly 460 also includes other metal components that are coupled to the metal enclosure 470, such as the metal washer 471, the metal endcap 472, or the like. In these embodiments, the outer edge of at least a portion of one or more other metal components form a portion of the metal enclosure 470. For instance, in some examples, the outer surface of metal endcap 472 forms a portion of the metal enclosure 470. These other metal components, such as metal washer 471 and metal endcap 472, may be composed of titanium or another suitable material. In some embodiments, at least some of these other metal components of the optical assembly 460 together form a metal base of the optical assembly 460. In at least some embodiments, the metal base acts as a metal housing for the non-conductive block 464. In at least some embodiments, the metal washer 471 and the metal endcap 472 are portions of a single component, such as a header. In at least some embodiments, the metal washer 471 is stepped, interfaces with metal enclosure 470, or provides a material backing for laser welding or any combination thereof. In at least some embodiments, the metal washer 471 and the metal endcap 472 (or either component alone) act as a metal housing for the non-conductive block 464.

The getter 441 includes a desiccant. Water vapor may build up on circuitry or other components within the optical assembly 460 and cause corrosion or other deleterious effects. In some embodiments, the getter 441 absorbs the water vapor, thereby improving the lifespan of the optical assembly 460. In some embodiments, rather than or in addition to the use of one or more getters or other desiccant, free space within the optical assembly 460 is filled or partially filled with an optically transparent substance other than air. In these embodiments, by taking the free space that would otherwise be occupied by air, vapor cannot build up, thereby reducing or preventing corrosion. For instance, in some examples, free space in the optical assembly 460 is filled or partially filled with a non-permeable transparent gel. In at least some embodiments, the gel is an optically transparent silicone gel or other suitable material.

The substrate 441 is composed at least partly of aluminum nitride. Aluminum nitride is a ceramic and is not electrically conductive. The light emitter(s) 435 are coupled to the substrate 441. In some embodiments, the light emitter(s) 435 are directly attached to the substrate 441. In other embodiments, the light emitter(s) 435 are coupled to the substrate 441 through one or more intermediaries, such as another substrate. In at least some embodiments, the substrate 441 is a substrate of a ceramic printed circuit board that is disposed within the optical assembly 460.

The substrate 441 is arranged within the metal enclosure 470 such that the aluminum nitride in the substrate 441 provides at least part of a thermal conductive path that conducts heat from the light emitter 435 to the metal enclosure 470. In at least some embodiments, the light emitter(s) 435 are directly attached to the substrate 441 and the substrate 441 directly touches the metal enclosure 470. In some embodiments, the substrate 441 is coupled to the light emitter(s) 435 through one or more intermediaries, which form a portion of the thermal conductive path. In some embodiments, there are one or more intermediaries in the thermal conductive path from the aluminum nitride in the substrate 441 and the metal enclosure 470, such as an adhesive, one or more intermediary metal components, or the like or any combination thereof.

In some embodiments, the enclosure size for the optical assembly 460 is determined based on the surgical tools being used for surgery. For instance, in some embodiments, the optical assembly 460 is used for deep brain surgery, with the enclosure size being based on the delivery cannula or other insertion tool used for deep brain surgery. In at least some embodiments, the optical assembly 460 emits light from the tip of the optical assembly 460.

Light emitters 435 generate heat, which may require the light emitter to be turned off for periods of time to cool. In at least some embodiments, the arrangement of the substrate 441 and the aluminum nitride in substrate 441 in the optical assembly 460 facilitates thermal transfer of heat way from the light emitter(s) 435 and heat dissipation for the optical assembly 460. In at least some embodiments, the arrangement of the substrate 441 and the aluminum nitride in substrate 441 in the optical assembly 460 can facilitate a longer emission period or less off-time for the light emitter(s) 435. In at least some embodiments, improved thermal transfer or heat dissipation facilitated by the aluminum nitride in the substrate 441 of the optical assembly 460 can reduce cycling of the light emitter 435.

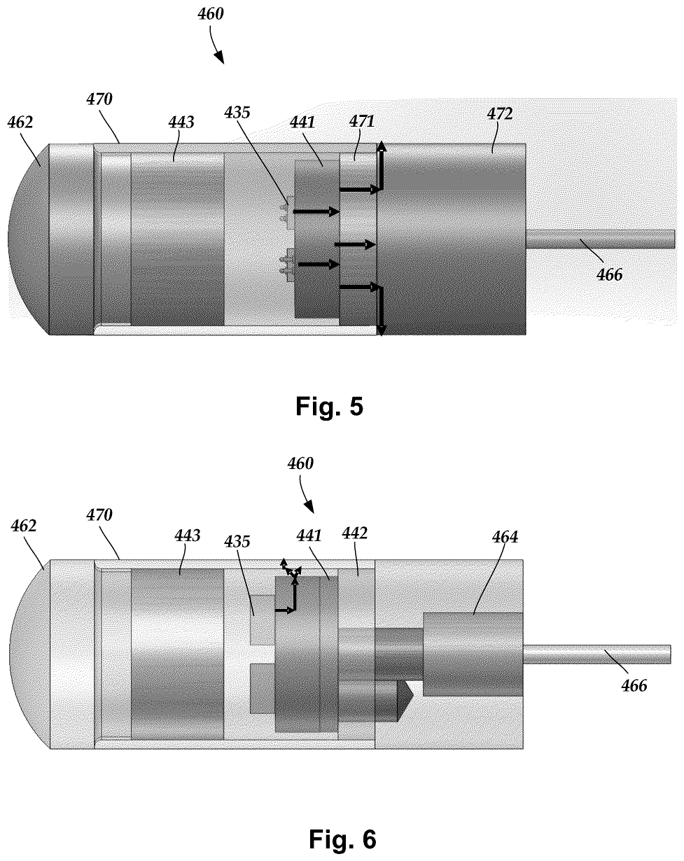

FIG. 5 illustrates another embodiment of an optical assembly 460, in which the substrate 441 directly touches a base of the optical assembly 460. The optical assembly 460 includes the light emitter 435, the emitter cover 462, the substrate 441, the optional seal 443, at least one electrical feedthrough pin 466, the metal enclosure 470, the metal washer 471, and the metal endcap 472. In at least some embodiments, the outer surface of the metal endcap 472 forms a portion of the metal enclosure 470. Together, the metal washer 471 and the metal endcap 472 form a metal base of the optical assembly 460. As discussed above, in at least some embodiments, metal washer 471 and metal endcap 472 are portions of a single component.

Although not shown in FIG. 5, in at least some embodiments, a non-conductive block (e.g., the non-conductive block 464 of FIG. 4) is disposed within the metal base. In some embodiments, an adhesive (for example, a thermally and electrically conductive epoxy) is used between the substrate 441 and the metal washer 471. In the embodiment illustrated in FIG. 5, during operation of the optical assembly 460, heat is conducted through the substrate 441, then conducted through the metal washer 471 and the metal endcap 472 and then dissipated by the tissue of the body of the patient in which the optical assembly 460 is implanted.

FIG. 6 illustrates a third embodiment of an optical assembly 460 in which in which a getter 442 is arranged between the substrate 441 and the non-conductive block 464. In FIG. 6, some elements are shown as partially transparent for illustrative purposes. In at least some embodiments, the optical assembly 460 includes the light emitter 435, the emitter cover 462, the substrate 441, the getter 442, the optional seal 443, the non-conductive block 464, at least one electrical feedthrough pin 466, and the metal enclosure 470. In the embodiment illustrated in FIG. 6, during operation of the optical assembly 460, heat is conducted through the substrate 441, and then through convention and radiation, the heat is transferred from the substrate 441 to the metal enclosure 470 and then dissipated by the tissue of the body of the patient in which the optical assembly 460 is implanted.

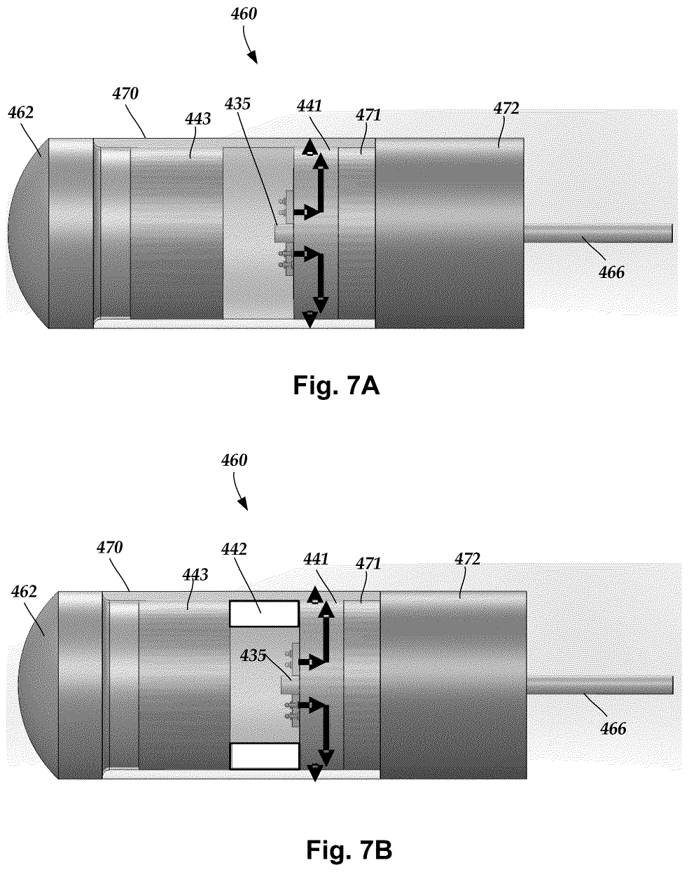

FIG. 7A illustrates a fourth embodiment of an optical assembly 460 in which the substrate 441 touches the metal enclosure 470. In at least some embodiments, the optical assembly 460 includes the light emitter 435, the emitter cover 462, the substrate 441, the optional seal 443, the metal enclosure 470, the metal washer 471, and the metal endcap 472. In the embodiment illustrated in FIG. 7A, during operation of the optical assembly 460, heat is conducted through the substrate 441, then conducted to the metal enclosure 470, and dissipated by the tissue of the body of the patient in which the optical assembly 460 is implanted.

FIG. 7B shows an embodiment of the optical assembly 460 of FIG. 7A that further includes a getter 442. In the embodiment illustrated in FIG. 7B, the getter 442 is shaped in a cylinder around the light emitter 435 and disposed (fully or partially) between the seal 443 and the substrate 441. The getter 442 includes a desiccant to absorb or otherwise remove water vapor in the free air space within the optical assembly 460.

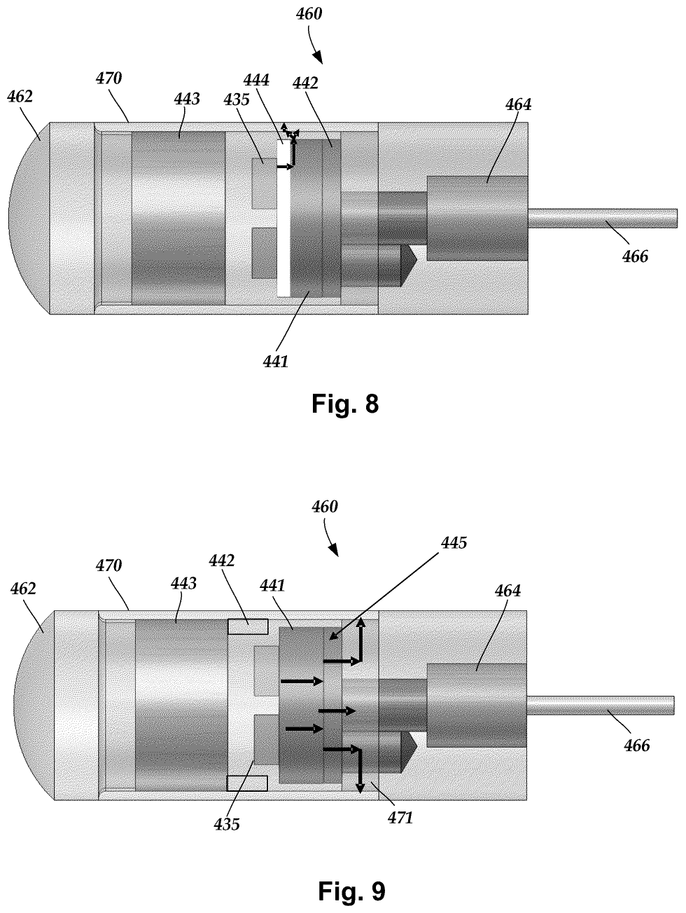

FIG. 8 illustrates a fifth embodiment of the optical assembly 460 in which in which the substrate 441 is disposed between a second substrate 444 and the getter 442. The second substrate 444 is different in composition from substrate 441. In FIG. 8, some elements are shown as partially transparent for illustrative purposes. In at least some embodiments, the optical assembly 460 includes the light emitter 435, the emitter cover 462, the second substrate 444, the substrate 441, the getter 442, the seal 443, the non-conductive block 464, at least one electrical feedthrough pin 466, and the metal enclosure 470. In at least some embodiments, the substrate 441 containing aluminum nitride is part of one printed circuit board that is disposed in optical assembly 540, and the second substrate 444 is part of a different printed circuit board that is disposed in the optical assembly 460. In at least some embodiments, the second substrate 444 is composed of polyimide.

In the embodiment illustrated in FIG. 8, during operation of the optical assembly 460, heat is conducted through the second substrate 444, then through the substrate 441 containing aluminum nitride to the metal enclosure 470 and then dissipated by the tissue of the body of the patient in which the optical assembly 460 is implanted. In the embodiment of the optical assembly 460 illustrated in FIG. 8, the light emitter 435 is connected directly to the second substrate 444 and the substrate 441 containing aluminum nitride is disposed between the second substrate 444 and getter 442. In some embodiments, it may be easier to connect the light emitter 435 directly to the second substrate 444 than directly to the substrate 441 containing aluminum nitride.

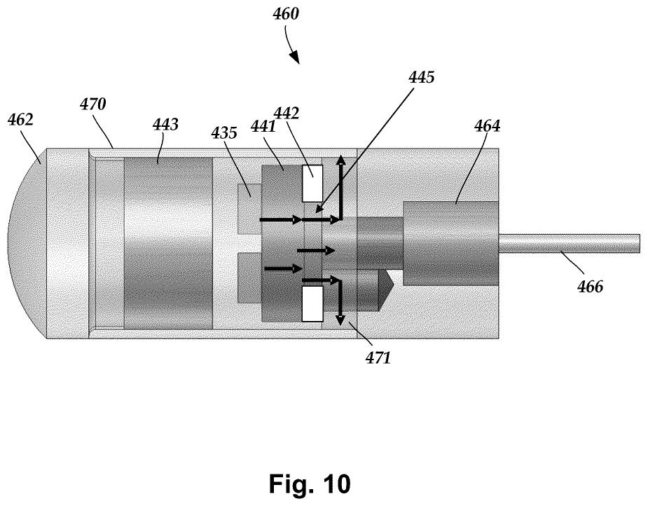

FIG. 9 illustrates a sixth embodiment of an optical assembly 460 in which an adhesive 445 that is thermally conductive is arranged between the substrate 441 and the metal washer 471. In FIG. 9, some elements are shown as partially transparent for illustrative purposes. In at least some embodiments, the optical assembly 460 includes the light emitter 435, the emitter cover 462, the substrate 441, the adhesive 445, the getter 442, the optional seal 443, the non-conductive block 464, at least one electrical feedthrough pin 466, the metal washer 471, and the metal enclosure 470.

In at least some embodiments, the adhesive 445 includes (for example, as a filler or additive) at least one thermally conductive material such as boron nitride. The thermally conductive material improves the thermal conductivity of the adhesive. For instance, in at least some embodiments, the adhesive 445 includes boron nitride mixed with polymer and epoxy for bonding to adjacent parts and to prevent air gaps. In at least some embodiments, the concentration of boron nitride in the adhesive 445 is in a range of 0.03 to 1.2 weight %. It will be understood that, under at least some circumstances, higher or lower concentrations of boron nitride may be suitable.

In the embodiment illustrated in FIG. 9, during operation of the optical assembly 460, heat is conducted through the substrate 441, then through the adhesive 445 the metal washer 471, then conducted to the metal enclosure 470, and dissipated by the tissue of the body of the patient in which the optical assembly 460 is implanted.

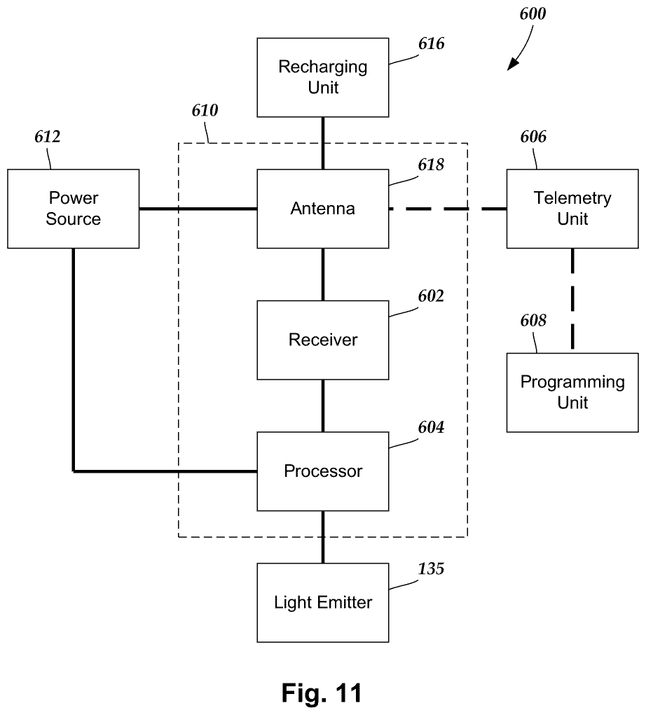

FIG. 10 illustrates a seventh embodiment of an optical assembly 460 in which in which an adhesive 445 is arranged between the substrate 441 and the metal washer 471, with the adhesive 445 being disposed within the getter 442. In FIG. 10, some elements are shown as partially transparent for illustrative purposes. In at least some embodiments, the optical assembly 460 includes the light emitter 435, the emitter cover 462, the substrate 441, the adhesive 445, the getter 442, the optional seal 443, the non-conductive block 464, at least one electrical feedthrough pin 466, the metal washer 471, and the metal enclosure 470.

In at least some embodiments, the getter 442 at least partially surrounds the adhesive 445. In at least some embodiments, the getter material in the getter 442 is pre-formed into a flat, thin ring, with the adhesive 445 being in the shape of a cylinder that is disposed within the ring formed by the getter 442. In these embodiments, this allows the boron nitride on the inside to provide thermal conduction while having the getter 442 on the outside provide moisture absorption. In the embodiment illustrated in FIG. 10, during operation of the optical assembly 460, heat is conducted through the substrate 441, then through the adhesive 445, the metal washer 471, and the metal enclosure 470, and dissipated by the tissue of the body of the patient in which the optical assembly 460 is implanted.

FIG. 11 is a schematic overview of one embodiment of components of an optical stimulation system 500 including an electronic subassembly 610 disposed within a control module. It will be understood that the optical stimulation system can include more, fewer, or different components and can have a variety of different configurations including those configurations disclosed in the stimulator references cited herein.

Some of the components (for example, a power source 612, an antenna 618, a receiver 602, and a processor 604) of the optical stimulation system can be positioned on one or more circuit boards or similar carriers within a sealed housing of an implantable pulse generator, if desired. Any power source 612 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 618 or a secondary antenna. The external power source can be in a device that is mounted on the skin of the user or in a unit that is provided near the user on a permanent or periodic basis.

If the power source 612 is a rechargeable battery, the battery may be recharged using the optional antenna 618, if desired. Power can be provided to the battery for recharging by inductively coupling the battery through the antenna to a recharging unit 616 external to the user. Examples of such arrangements can be found in the references identified above.

In one embodiment, light is emitted by the light emitter 135 of the lead body to stimulate nerve fibers, muscle fibers, or other body tissues near the optical stimulation system. The processor 604 is generally included to control the timing and other characteristics of the optical stimulation system. For example, the processor 604 can, if desired, control one or more of the timing, pulse frequency, strength, duration, and waveform of the optical stimulation. In addition, the processor 604 can select one or more of the optional electrodes to provide electrical stimulation, if desired. In some embodiments, the processor 604 selects which of the optional electrode(s) are cathodes and which electrode(s) are anodes.

Any processor can be used and can be as simple as an electronic device that, for example, produces optical stimulation at a regular interval or the processor can be capable of receiving and interpreting instructions from an external programming unit 608 that, for example, allows modification of stimulation characteristics. In the illustrated embodiment, the processor 604 is coupled to a receiver 602 which, in turn, is coupled to the optional antenna 618. This allows the processor 604 to receive instructions from an external source to, for example, direct the stimulation characteristics and the selection of electrodes, if desired.

In one embodiment, the antenna 618 is capable of receiving signals (e.g., RF signals) from an external telemetry unit 606 which is programmed by the programming unit 608. The programming unit 608 can be external to, or part of, the telemetry unit 606. The telemetry unit 606 can be a device that is worn on the skin of the user or can be carried by the user and can have a form similar to a pager, cellular phone, or remote control, if desired. As another alternative, the telemetry unit 606 may not be worn or carried by the user but may only be available at a home station or at a clinician's office. The programming unit 608 can be any unit that can provide information to the telemetry unit 606 for transmission to the optical stimulation system 600. The programming unit 608 can be part of the telemetry unit 606 or can provide signals or information to the telemetry unit 606 via a wireless or wired connection. One example of a suitable programming unit is a computer operated by the user or clinician to send signals to the telemetry unit 606.

The signals sent to the processor 604 via the antenna 618 and the receiver 602 can be used to modify or otherwise direct the operation of the optical stimulation system. For example, the signals may be used to modify the stimulation characteristics of the optical stimulation system such as modifying one or more of stimulation duration, pulse frequency, waveform, and stimulation amplitude. The signals may also direct the optical stimulation system 600 to cease operation, to start operation, to start charging the battery, or to stop charging the battery. In other embodiments, the stimulation system does not include the antenna 618 or receiver 602 and the processor 604 operates as programmed.

Optionally, the optical stimulation system 600 may include a transmitter (not shown) coupled to the processor 604 and the antenna 618 for transmitting signals back to the telemetry unit 606 or another unit capable of receiving the signals. For example, the optical stimulation system 600 may transmit signals indicating whether the optical stimulation system 600 is operating properly or not or indicating when the battery needs to be charged or the level of charge remaining in the battery. The processor 604 may also be capable of transmitting information about the stimulation characteristics so that a user or clinician can determine or verify the characteristics.

It is intended that the terminology used in this disclosure be interpreted in its broadest reasonable manner, even though it is being used in conjunction with a detailed description of certain examples of the technology. Although certain terms may be emphasized below, any terminology intended to be interpreted in any restricted manner will be overtly and specifically defined as such in this Detailed Description section. Throughout the specification and claims, the following terms take at least the meanings explicitly associated herein, unless the context dictates otherwise. The meanings identified below do not necessarily limit the terms, but merely provide illustrative examples for the terms. For example, each of the terms “based on” and “based upon” is not exclusive, and is equivalent to the term “based, at least in part, on,” and includes the option of being based on additional factors, some of which may not be described herein. As another example, the term “via” is not exclusive, and is equivalent to the term “via, at least in part,” and includes the option of being via additional factors, some of which may not be described herein. The meaning of “in” includes “in” and “on.” The phrases “in one embodiment,” “in one example,” “in some examples,” “in some embodiments,” “in at least some embodiments,” as used herein does not necessarily refer to the same embodiment(s) or example(s), although it may. Use of particular textual numeric designators does not imply the existence of lesser-valued numerical designators. References in the singular are made merely for clarity of reading and include plural references unless plural references are specifically excluded. The term “or” is an inclusive “or” operator unless specifically indicated otherwise. For example, the phrases “A or B” means “A, B, or A and B.” As used herein, the terms “component” and “system” are intended to encompass hardware, software, or various combinations of hardware and software. Thus, for example, a system or component may be a process, a process executing on a computing device, the computing device, or a portion thereof.

The above description provides specific details for a thorough understanding of, and enabling description for, various examples of the structure, manufacture, and use of the technology. One skilled in the art will understand that the technology may be practiced without many of these details. In some instances, well-known structures and functions have not been shown or described in detail to avoid unnecessarily obscuring the description of examples of the technology. Also, since many embodiments of the invention can be made without departing from the spirit and scope of the invention, the invention also resides in the claims hereinafter appended.

What is claimed as new and desired to be protected by Letters Patent of the United States is:

1. An optical stimulation lead, comprising:
a lead body comprising a distal end, a distal portion, and a proximal portion; and
an optical assembly attached to the distal end of the lead body, wherein the optical assembly comprises:
a metal enclosure;
a light emitter disposed within the metal enclosure;
an electrical feedthrough pin electrically coupled to the light emitter;
a first substrate disposed within the metal enclosure and coupled to the light emitter, wherein the first substrate comprises aluminum nitride, wherein the first substrate is arranged within the metal enclosure with the aluminum nitride in a thermal conductive path that conducts heat from the light emitter to the metal enclosure; and
an emitter cover disposed opposite the lead body and configured to transmit light from the light emitter, wherein the emitter cover is coupled to the metal enclosure.

2. The optical stimulation lead of claim 1, wherein the metal enclosure comprises titanium.

3. The optical stimulation lead of claim 1, wherein the light emitter comprises a laser diode.

4. The optical stimulation lead of claim 1, wherein the optical assembly forms a hermetically sealed housing for the light emitter.

5. The optical stimulation lead of claim 1, further comprising a plurality of terminals disposed along the proximal portion of the lead body and a plurality of conductors extending along the lead body, wherein at least one of the conductors of the plurality of conductors electrically couples the electrical feedthrough pin to at least one of the terminals of the plurality of terminals of the lead.

6. The optical stimulation lead of claim 1, wherein the emitter cover comprises at least one of an optical diffuser or a lens.

7. The optical stimulation lead of claim 1, wherein the optical assembly further comprises a non-permeable gel disposed within the metal enclosure.

8. The optical stimulation lead of claim 1, wherein the optical assembly further comprises a second substrate different from the first substrate, wherein the light emitter is attached to the second substrate and the second substrate is disposed between the first substrate and the light emitter.

9. The optical stimulation lead of claim 1, wherein the optical assembly further comprises a getter disposed within the metal enclosure, wherein the getter comprises a desiccant.

10. The optical stimulation lead of claim 9, wherein the first substrate is disposed between the light emitter and the getter.

11. The optical stimulation lead of claim 9, wherein the getter is disposed on the metal enclosure around the light emitter.

12. The optical stimulation lead of claim 1, wherein the optical assembly further comprises:
a non-conductive block, wherein the feedthrough pin extends through the non-conductive block; and
a metal base disposed around the non-conductive block.

13. The optical stimulation lead of claim 12, wherein the first substrate is in contact with the metal base.

14. The optical stimulation lead of claim 1, wherein the optical assembly further comprises an adhesive, wherein the adhesive comprises boron nitride and the first substrate is disposed between the light emitter and the adhesive.

15. The optical stimulation lead of claim 14, wherein the optical assembly further comprises a getter disposed within the metal enclosure, wherein the getter comprises a desiccant, the first substrate is disposed between the light emitter and the getter, and the getter at least partially surrounds the adhesive.

16. An optical stimulation system, comprising:
the optical stimulation lead of claim 1; and
a control module coupleable to the optical stimulation lead, the control module comprising
a housing, and
an electronic subassembly disposed in the housing and configured and arranged to provide electrical signals to the light emitter of the optical stimulation lead.

17. The optical stimulation system of claim 16, wherein the optical stimulation lead further comprises a plurality of terminals disposed along the proximal portion of the lead body, the optical stimulation system further comprising a connector for receiving the proximal portion of the lead body, the connector having a proximal end, a distal end, and a longitudinal length, the connector comprising:
a connector housing defining a port at the distal end of the connector, the port configured and arranged for receiving the proximal portion of the lead body of the optical stimulation lead, and
a plurality of connector contacts disposed in the connector housing, the plurality of connector contacts configured and arranged to couple to the plurality of terminals disposed along the proximal portion of the lead body of the optical stimulation lead.

18. The electrical stimulation system of claim 17, wherein the connector is part of the control module.

19. The electrical stimulation system of claim 17, further comprising a lead extension coupleable to both the electrical stimulation lead and the control module, wherein the connector is part of the lead extension.

20. A method of stimulating patient tissue, the method comprising:
implanting the optical stimulation lead of claim 1 into the patient tissue; and
emitting light from the light emitter of the optical stimulation lead to stimulate the patient tissue.
